SEQUENCE LISTING (31 sequences)

SEQ ID NO: 1 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 2 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 3 - DNA, 1500 bp, Synthetic construct
  source 1..1500: mol_type = other DNA
atgattcttaaaattctgaacgaaatagcatctattggttcaactaaacagaagcaagcaattcttaaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 4 - protein, 499 aa, Synthetic construct
MILKILNEIASIGSTKQKQAILKKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 5 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattaaagaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 6 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIKELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 7 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtgaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 8 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGEKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 9 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctaaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 10 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPKQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 11 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgataaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 12 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDKKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 13 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgataaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 14 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDKLDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 15 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctaaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 16 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPKNSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 17 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctaaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 18 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISKKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 19 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctgaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 20 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSELEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 21 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattaaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 22 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIKNQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 23 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattcgtcaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 24 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIRQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 25 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattcgtgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 26 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIRVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGSGSSGHHHHHH

SEQ ID NO: 27 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctaaaatgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 28 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILKCECNGWLKSDGRTDYVKLFLPIAIRLREDKTKANTFEDVFGDFHEVTGLGSGSSGHHHHHH

SEQ ID NO: 29 - DNA, 1500 bp, Synthetic construct
  source 1..1500: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcaaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggttctggcagttcaggtcatcaccaccatcatcactaa

SEQ ID NO: 30 - protein, 499 aa, Synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECKCNGWLKSDGRTDYVKLFLPIAIRLREDKTKANTFEDVFGDFHEVTGLGSGSSGHHHHHH

SEQ ID NO: 31 - protein, 14 aa, Synthetic construct
GLGSGSSGHHHHHH

(19) United States
(12) Patent Application Publication
(10) Pub. No.: US 2023/0212550 A1
(43) Pub. Date: Jul. 6, 2023

(54) T4 DNA Ligase Variants with Increased Ligation Efficiency

(71) Applicant: AbClonal Science, Inc., Woburn, MA (US)

(72) Inventors: Zhenyu Zhu, Lynnfield, MA (US); Aine Quimby, Newburyport, MA (US); Dapeng Sun, Lexington, MA (US); Alicia DiCicco, Beverly, MA (US)

(21) Appl. No.: 18/073,598

(22) Filed: Dec. 2, 2022

Related U.S. Application Data
(60) Provisional application No. 63/285,137, filed on Dec. 2, 2021.

(51) Int. Cl. C12N 9/00

(52) U.S. Cl. CPC C12N 9/93

(57) ABSTRACT

The invention includes a mutant T4 DNA ligase or a biologically active fragment thereof, which has greater activity than wild type T4 DNA ligase. The mutant T4 DNA ligase, or the biologically active fragment, has one or more substitutions differing from the wild type, as described more fully in the Summary with reference to the Sequence Listing.

### BACKGROUND

Ligases are commonly used in molecular biology for forming phosphodiester bonds between duplex nucleic acid fragments at the intersection of juxtaposed 5′ phosphate and 3′ hydroxyl termini. By designing complementary overhangs between each of the double-stranded fragments, ligation can be directed to be both positionally specific and directionally oriented. This allows for specific integration of DNA or RNA fragments into larger vectors, as needed in most molecular biology research.

Of the commercially available ligases, T4 DNA Ligase is a versatile enzyme that catalyzes the bond joining duplex DNA or RNA at both cohesive ends and blunt ends, has a rapid ligation speed, and also repairs via ligation the mismatches that exist in nicked DNA. It can also ligate either cohesive or blunt ends of DNA, RNA and RNA-DNA hybrids (but not single-stranded nucleic acids). It can also ligate blunt-ended DNA with much greater efficiency than E. coli DNA ligase.

Because T4 DNA Ligase is the backbone of many molecular biology protocols and is in constant demand, it generates a large share of the revenue for many life science products companies due to its significance in the molecular biology reagent market. Increasing the activity of T4 DNA ligase has benefits for manufacturing of the T4 DNA ligase enzyme itself, allowing fewer production runs to be necessary to create significant final product. Increasing the activity of T4 DNA ligase also increases the usefulness of the enzyme by requiring less enzyme or less total time for a complete ligation of substrates, depending on the goal of the user.

### SUMMARY

The invention relates to engineered T4 DNA Ligase mutants exhibiting enhanced ligation activity compared to the wild-type T4 DNA ligase. The following T4 DNA ligase mutants (with a substitution at the position indicated and wherein each mutant's amino acid sequence is in the sequence identification number following the indicated substitution, except that in the sequence listing sequences, each mutant also includes the tag sequence at their C-terminus: GLGSGSSGHHHHHH (SEQ ID NO: 32); and wherein a DNA sequence for each such mutant is also shown as the odd-numbered sequence identification number in the sequence listing preceding the amino acid sequence identification number of each mutant) were identified as having such enhanced ligation activity: E23K (SEQ ID NO: 4), E88K (SEQ ID NO: 6), K98E (SEQ ID NO: 8), E132K (SEQ ID NO: 10), E143K (SEQ ID NO: 12), E173K (SEQ ID NO: 14), E240K (SEQ ID NO: 16), E271K (SEQ ID NO: 18), K306E (SEQ ID NO: 20), E321K (SEQ ID NO: 22), D340R (SEQ ID NO: 24), D371R (SEQ ID NO: 26), E438K (SEQ ID NO: 28) and E440K (SEQ ID NO: 30).

The invention further includes T4 DNA ligase mutant amino acid sequences with at least one of the mutations above, but wherein the remainder of the T4 DNA ligase mutant amino acid sequence only has conservative substitutions such that the molecule has at least 70%, 80%, 90%, 95%, 96%, 97%, 98% or 99% identity to the corresponding T4 DNA ligase mutant amino acid sequence (but without the tag sequence (SEQ ID NO: 32)) in the sequence listing (hereinafter referred to as “Variant Sequences”).

The invention further includes the DNA sequences identified by the odd numbered sequence preceding each of the amino acid sequences for the mutants above (i.e., respectively, SEQ ID NOS: 3; 5; 7; 9; 11; 12; 13; 15; 17; 19; 21; 23; 25; 27; and 29, but without the 5′ terminal portion of each sequence encoding the tag sequence) and further includes the foregoing DNA sequences and other degenerate nucleic acid sequences (collectively the “Degenerate Nucleic Acid Sequences”) encoding (i) each of the above T4 DNA ligase mutants, and (ii) each of the amino acid sequences of any of the Variant Sequences.

The invention further includes vectors incorporating any Degenerate Nucleic Acid Sequences; and cells transformed with any such vectors or Degenerate Nucleic Acid Sequences and capable of expressing any of the above T4 DNA ligase mutant amino acid sequences or Variant Sequences.

The invention further includes a composition or a kit comprising any of the above T4 DNA ligase mutant amino acid sequences or Variant Sequences, Degenerate Nucleic Acid Sequences, or vectors incorporating such Degenerate Nucleic Acid Sequences. The invention also includes a process of amplifying a target nucleic acid, wherein any of the above T4 DNA ligase mutants or Variant Sequences are employed in a reaction mixture designed to amplify a target nucleic acid, and subjecting the reagent mixture to conditions for amplification of the target nucleic acid.

The above mutant T4 DNA ligase mutants have greater activity at lower concentrations in amplifying target DNA sequences compared with wild type, and the Variant Sequences are also expected to have such greater activity.

### BRIEF DESCRIPTION OF THE DRAWINGS

FIGS. 1A, 1B show a series of gel electrophoresis results from an activity assay of the wild type T4 DNA ligase (“WT” at top left) compared with each of the T4 DNA ligase mutants. There are 12 columns in each gel, such that left to right, each column represents the concentration of T4 DNA ligase following a 1:2 serial dilution, and wherein in column 1, there was 700 ng of T4 DNA Ligase (or mutant) initially present in the solution. Each gel has an arrow at the dilution level where significant amounts of (upper band, labeled “1” on right) supercoiled plasmid product and restriction digested linearized substrate plasmid (middle band, labeled “2” on right) are apparent. The lowermost band (labeled “3” on right) is the open-circle plasmid product ligated by the T4 DNA ligase wild type or the mutant indicated.

### DETAILED DESCRIPTION

The term “biologically active fragment” refers to any fragment, derivative, homolog or analog of a T4 DNA ligase mutant that possesses in vivo or in vitro activity that is characteristic of that biomolecule; including, for example, ligase activity or repairing via ligation the mismatches that exist in nicked DNA. In some embodiments, the biologically active fragment, derivative, homolog or analog of the mutant T4 DNA ligase possesses any degree of the biological activity of the mutant T4 DNA ligase in any in vivo or in vitro assay of interest.

In some embodiments, the biologically active fragment can optionally include any number of contiguous amino acid residues of the mutant T4 DNA ligase. The invention also includes the polynucleotides encoding any such biologically active fragment.

Biologically active fragments can arise from post transcriptional processing or from translation of alternatively spliced RNAs, or alternatively can be created through engineering, bulk synthesis, or other suitable manipulation. Biologically active fragments include fragments expressed in native or endogenous cells as well as those made in expression systems such as, for example, in bacterial, yeast, plant, insect or mammalian cells.

As used herein, the phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz (1979) Principles of Protein Structure, Springer-Verlag). According to such analyses, groups of amino acids can be defined where amino acids within a group exchange preferentially with each other, and therefore resemble each other most in their impact on the overall protein structure (Schulz (1979) supra). Examples of amino acid groups defined in this manner can include: a “charged/polar group” including Glu, Asp, Asn, Gln, Lys, Arg, and His; an “aromatic or cyclic group” including Pro, Phe, Tyr, and Trp; and an “aliphatic group” including Gly, Ala, Val, Leu, Ile, Met, Ser, Thr, and Cys. Within each group, subgroups can also be identified. For example, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His, and Trp; and the “phenyl sub-group” comprising Phe and Tyr. In another further example, the aliphatic group can be sub-divided into sub-groups including: the “large aliphatic non-polar sub-group” comprising Val, Leu, and Ile; the “aliphatic slightly-polar sub-group” comprising Met, Ser, Thr, and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH2 can be maintained. A “conservative variant” is a polypeptide that includes one or more amino acids that have been substituted to replace one or more amino acids of the reference polypeptide (for example, a polypeptide whose sequence is disclosed in a publication or sequence database, or whose sequence has been determined by nucleic acid sequencing) with an amino acid having common properties, e.g., belonging to the same amino acid group or sub-group as delineated above.

When referring to a gene, “mutant” means the gene has at least one base (nucleotide) change, deletion, or insertion with respect to a native or wild type gene. The mutation (change, deletion, and/or insertion of one or more nucleotides) can be in the coding region of the gene or can be in an intron, 3′ UTR, 5′ UTR, or promoter region. As nonlimiting examples, a mutant gene can be a gene that has an insertion within the promoter region that can either increase or decrease expression of the gene; can be a gene that has a deletion, resulting in production of a nonfunctional protein, truncated protein, dominant negative protein, or no protein; or, can be a gene that has one or more point mutations leading to a change in the amino acid of the encoded protein or results in aberrant splicing of the gene transcript.

The terms “mutant T4 DNA ligase of the invention” and “mutant T4 DNA ligase” when used in this Detailed Description section refer to, depending on the context, collectively or individually, the mutant T4 DNA Ligase polypeptides tested and exhibiting enhanced ligation activity which are:

(SEQ ID NO: 4)
E23K,
(SEQ ID NO: 6)
E88K,
(SEQ ID NO: 8)
K98E,
(SEQ ID NO: 10)
E132K,
(SEQ ID NO: 12)
E143K,
(SEQ ID NO: 14)
E173K,
(SEQ ID NO: 16)
E240K,
(SEQ ID NO: 18)
E271K,
(SEQ ID NO: 20)
K306E,
(SEQ ID NO: 22)
E321K,
(SEQ ID NO: 24)
D340R,
(SEQ ID NO: 26)
D371R,
(SEQ ID NO: 28)
E438K
and
(SEQ ID NO: 30)
E440K;

and/or Variant Sequences and/or Degenerate Nucleic Acid Sequences, as those terms are defined in the Summary section.

“Naturally-occurring” or “wild-type” refers to the form found in nature. For example, a naturally occurring or wild-type polypeptide or polynucleotide sequence is a sequence present in an organism, which has not been intentionally modified by human manipulation.

The terms “percent identity” or “homology” with respect to nucleic acid or polypeptide sequences are defined as the percentage of nucleotide or amino acid residues in the candidate sequence that are identical with the known polypeptides, after aligning the sequences for maximum percent identity and introducing gaps, if necessary, to achieve the maximum percent homology. N-terminal or C-terminal insertion or deletions shall not be construed as affecting homology. Homology or identity at the nucleotide or amino acid sequence level can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268), which are tailored for sequence similarity searching. The approach used by the BLAST program is to first consider similar segments, with and without gaps, between a query sequence and a database sequence, then to evaluate the statistical significance of all matches that are identified, and finally to summarize only those matches which satisfy a preselected threshold of significance. For a discussion of basic issues in similarity searching of sequence databases, see Altschul (1994), Nature Genetics 6, 119-129. The search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx is the BLOSUM62 matrix (Henikoff (1992), Proc. Natl. Acad. Sci. USA 89, 10915-10919), recommended for query sequences over 85 units in length (nucleotide bases or amino acids).

In some embodiments, the invention relates to methods (and related kits, systems, apparatuses and compositions) for performing a ligation reaction comprising or consisting of contacting a mutant T4 DNA ligase or a biologically active fragment thereof with a nucleic acid template in the presence of one or more nucleotides, and ligating at least one of the one or more nucleotides using the mutant T4 DNA ligase or the biologically active fragment thereof.

In some embodiments, the method can include ligating a double stranded RNA or DNA polynucleotide strand into a circular molecule. In some embodiments, the method can further include detecting a signal indicating the ligation by using a sensor. In some embodiments, the sensor is an ISFET. In some embodiments, the sensor can include a detectable label or detectable reagent within the ligating reaction.

In some embodiments, the invention relates to methods (and related kits, systems, apparatus and compositions) for performing rolling circle amplification (see U.S. Pat. No. 5,714,320, incorporated by reference) of a nucleic acid, using the mutant T4 DNA ligase as the enzyme in the ligation steps of the amplification process. The amplifying includes amplifying the nucleic acid in solution, as well as clonally amplifying the nucleic acid on a solid support such as a nucleic acid bead, flow cell, nucleic acid array, or wells present on the surface of the solid support.

Making Mutant T4 DNA Ligase

The mutant T4 DNA ligase of the invention can be expressed in any suitable host system, including a bacterial, yeast, fungal, baculovirus, plant or mammalian host cell. For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xy1A and xy1B genes, and prokaryotic beta-lactamase gene (Villa-Kamaroff et al., 1978, Proc. Natl Acad. Sci. USA 75: 3727-3731), as well as the tac promoter (DeBoer et al., 1983, Proc. Natl Acad. Sci. USA 80: 21-25).

For filamentous fungal host cells, suitable promoters for directing the transcription of the nucleic acid constructs of the present disclosure include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-488.

For baculovirus expression, insect cell lines derived from Lepidopterans (moths and butterflies), such as Spodoptera frugiperda, are used as host. Gene expression is under the control of a strong promoter, e.g., pPolh.

Plant expression vectors are based on the Ti plasmid of Agrobacterium tumefaciens, or on the tobacco mosaic virus (TMV), potato virus X, or the cowpea mosaic virus. A commonly used constitutive promoter in plant expression vectors is the cauliflower mosaic virus (CaMV) 35S promoter.

For mammalian expression, cultured mammalian cell lines such as the Chinese hamster ovary (CHO), COS, including human cell lines such as HEK and HeLa may be used to produce the mutant T4 DNA ligase. Examples of mammalian expression vectors include the adenoviral vectors, the pSV and the pCMV series of plasmid vectors, vaccinia and retroviral vectors, as well as baculovirus. The promoters for cytomegalovirus (CMV) and SV40 are commonly used in mammalian expression vectors to drive gene expression. Non-viral promoters, such as the elongation factor (EF)-1 promoter, are also known.

The control sequence for the expression may also be a suitable transcription terminator sequence, that is, a sequence recognized by a host cell to terminate transcription. The terminator sequence is operably linked to the 3′ terminus of the nucleic acid sequence encoding the polypeptide. Any terminator which is functional in the host cell of choice may be used.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase.

Terminators for insect, plant and mammalian host cells are also well known.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiol Rev 57: 109-137.

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells can be from the genes for Saccharomyces cerevisiae alpha-factor and Saccharomyces cerevisiae invertase. Signal peptides for other host cell systems are also well known.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the mutant T4 DNA ligase relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter. Regulatory systems for other host cells are also well known.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the KRED polypeptide of the present invention would be operably linked with the regulatory sequence.

Another embodiment includes a recombinant expression vector comprising a polynucleotide encoding an engineered mutant T4 DNA ligase or a variant thereof, and one or more expression regulating regions such as a promoter and a terminator, and a replication origin, depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the mutant T4 DNA ligase at such sites. Alternatively, the nucleic acid sequences of the mutant T4 DNA ligase may be expressed by inserting the nucleic acid sequences or a nucleic acid construct comprising the sequences into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the mutant T4 DNA ligase polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector herein preferably contain one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, METS, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus. Selectable markers for insect, plant and mammalian cells are also well known.

The expression vectors of the present invention preferably contain an element(s) that permits integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A ori, or the origins of replication of plasmids pBR322, pUC19, pACYC177 (which plasmid has the P15A ori), or pACYC184 permitting replication in E. coli, and pUB110, pE194, pTA1060, or pAM31 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes it's functioning temperature-sensitive in the host cell (see, e.g., Ehrlich, 1978, Proc Natl Acad Sci. USA 75:1433).

More than one copy of a nucleic acid sequence of the mutant T4 DNA ligase may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Expression vectors for the mutant T4 DNA ligase polynucleotide are commercially available. Suitable commercial expression vectors include p3xFLAGTM expression vectors from Sigma-Aldrich Chemicals, St. Louis Mo., which includes a CMV promoter and hGH polyadenylation site for expression in mammalian host cells and a pBR322 origin of replication and ampicillin resistance markers for amplification in E. coli. Other suitable expression vectors are pBluescriptll SK(−) and pBK-CMV, which are commercially available from Stratagene, LaJolla Calif., and plasmids which are derived from pBR322 (Gibco BRL), pUC (Gibco BRL), pREP4, pCEP4 (Invitrogen) or pPoly (Lathe et al., 1987, Gene 57:193-201).

Suitable host cells for expression of a polynucleotide encoding the mutant T4 DNA ligase, are well known in the art and include but are not limited to, bacterial cells, such as E. coli, Lactobacillus kefir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture mediums and growth conditions for the above-described host cells are well known in the art. Polynucleotides for expression of the mutant T4 DNA ligase may be introduced into cells by various methods known in the art. Techniques include among others, electroporation, biolistic particle bombardment, liposome mediated transfection, calcium chloride transfection, and protoplast fusion. Various methods for introducing polynucleotides into cells are known to the skilled artisan.

Polynucleotides encoding the mutant T4 DNA ligase can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides can be prepared by chemical synthesis using, e.g., the classical phosphoramidite method described by Beaucage et al., 1981, Tet Lett 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., and Operon Technologies Inc., Alameda, Calif.

Engineered mutant T4 DNA ligase expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B.TM. from Sigma-Aldrich of St. Louis Mo.

Chromatographic techniques for isolation of the mutant T4 DNA ligase include, among others, reverse phase chromatography, high performance liquid chromatography, ion exchange chromatography, gel electrophoresis, and affinity chromatography. Conditions for purification will depend, in part, on factors such as net charge, hydrophobicity, hydrophilicity, molecular weight, molecular shape, and will be apparent to those having skill in the art.

In some embodiments, affinity techniques may be used to isolate the mutant T4 DNA ligase. For affinity chromatography purification, any antibody which specifically binds the mutant T4 DNA ligase may be used. For the production of antibodies, various host animals, including but not limited to rabbits, mice, rats, etc., may be immunized by injection with a compound. The compound may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette Guerin) and Corynebacterium parvum.

Example of Making T4 DNA Ligase Mutants

T4 DNA ligase mutants were generated by conventional PCR mutagenesis wherein the primers are designed to include the desired base substitution, and during PCR, the mutation is incorporated into the amplicon, replacing the original sequence. All T4 DNA ligase mutants and the wild type had an added C-terminal 6-mer His tag for ease of purification, preceded by a 6-mer series of Ser and Gly residues as shown.

PCR was followed with a DpnI digestion which destroys the methylated template (that doesn't contain the substitution) leaving behind only the unmethylated PCR amplicons with the substitution.

The PCR amplicons are then then directly transformed into chemically competent E. Coli host cells, wherein the bacteria were pre-treated with chemicals to enable them to take up plasmids incorporating the amplicons. See ThermoFisher Scientific, Chemically Competent Cells webpage (featuring kits for generating chemically competent cells).

The mutant T4 DNA ligase polypeptides expressed from the transformed E. Coli host cells were characterized and selected based on a standard ligation assay with gel electrophoresis. Ligase catalyzes the formation of phosphodiester bonds between the 5′ and 3′ ends of complementary cohesive ends or blunt ends of duplex DNA, and the extent of ligation with different T4 DNA ligase mutants can be visualized on an agarose gel using appropriate DNA dyes. In this case, GelRed (Biotium, San Francisco Calif.) was used to stain the gels for visualization under UV light. Examining each T4 DNA ligase mutant's performance based on their ligation activity at diminishing concentrations of enzyme, and comparing the resultant activity to that of similarly diluted wild type (“WT”) ligase, allowed determination of which mutants showed increased activity compared to the wild-type under the same conditions.

The ligation substrate for characterization of the mutant T4 DNA ligase polypeptides expressed from the transformed E. Coli host cells was prepared as follows.

The DNA vector used was pUC19 (New England Bio Labs, catalog number N3041S). PUC19 is a double stranded circle that is 2686 base pairs long. PUC19 was digested with BsaI-HF® v2 (New England Bio Labs, catalog number R3733S) which has an offset/nonsymmetrical cleavage site, where the 5′ strand is cleaved after the recognition sequence plus the addition of one random (N1) nucleotide, and on the 3′ strand cleavage occurs after the complementary recognition sequence plus five additional random (N5) nucleotides. The 5′ recognition sequence is GGTCTC (SEQ ID NO: 31). The cleavage site of PUC19 by BsaI-HF® v2 is designated 5′-GGTCTC(N1)/(N5)-3′.

5 μl of puC19 at a concentration of 1 mg/ml was combined with 2.5 μl of 20,000 units/ml BsaI-HF® v2, 5 μl 10× rCutSmart™ Buffer (New England Bio Labs, catalog number B6004S) (50 mM Potassium Acetate, 20 mM Tris-acetate, 10 mM Magnesium Acetate, 100 μg/ml Recombinant Albumin), and 35 μl of water. After all components were combined, the composition was incubated at 37° C. for the digestion to take place. After one hour at 37° C., the reaction was incubated at 80° C. for 20 minutes to heat inactivate the BsaI-HF® v2. The mixture was then diluted with water to a concentration of 10 ng/μl.

### Example: Ligation Assay and Results

The ligation procedure was performed as follows. Each T4 DNA ligase either wild-type or variant was diluted with enzyme diluent (50% glycerol, 10 mM tris-HCl) in a serial dilution such that for each sample 10 different concentrations were obtained. The starting concentration for each serial dilution was 100 ng/ul, diluted two fold in each next dilution such that the final concentration was 50% as concentrated as the sample before it, and so on for a total of 10 1:2 dilutions. Seven μl of each enzyme or serial dilution was then added to a PCR plate such that the approximate amount of the enzyme in lanes 1 to lane 10 is, respectively, 700, 350, 175, 88, 44, 22, 11, 5.5, 2.7, 1.4 ng. The 11 and 12 lanes have no mutant or wild type T4 DNA ligase added to the reaction, showing how far the reaction travels when no ligation occurs.

To the enzyme, 13 μl master mix consisting of 2 μl of 10×T4 DNA Ligase Reaction Buffer (New England Biolabs, catalog number B0202A; including 50 mM Tris-HCl, 10 mM MgCl₂, 1 mM ATP, 10 mM DTT (dithiothreitol)), 1 μl of 10 ng/μl pUC19 digested with BsaI-HF® v2, and 10 μl of water was added to each reaction. This makes the total volume per reaction up to 23 μl. The reactions were incubated at 16° C. for 30 minutes. After the 30-minute incubation period the reactions received a heat shock of 80° C. for 2 minutes to stop any further activity. 4 μl of stop solution (120 mM EDTA, 30% glycerol, 50 mM Tris-HCl pH 8.0, 0.0125% bromophenol blue, 0.1% SDS, and 5×Gel Red Nucleic Acid Stain (Biotium, Fremont, Calif.)) was added to each reaction.

Gel electrophoresis with 8% agarose gel was used to visualize the ligation reaction product. Each gel had a wild-type T4 DNA ligase sample series and 7 variant T4 DNA ligase sample series. Each gel was run for 25 minutes at 200V.

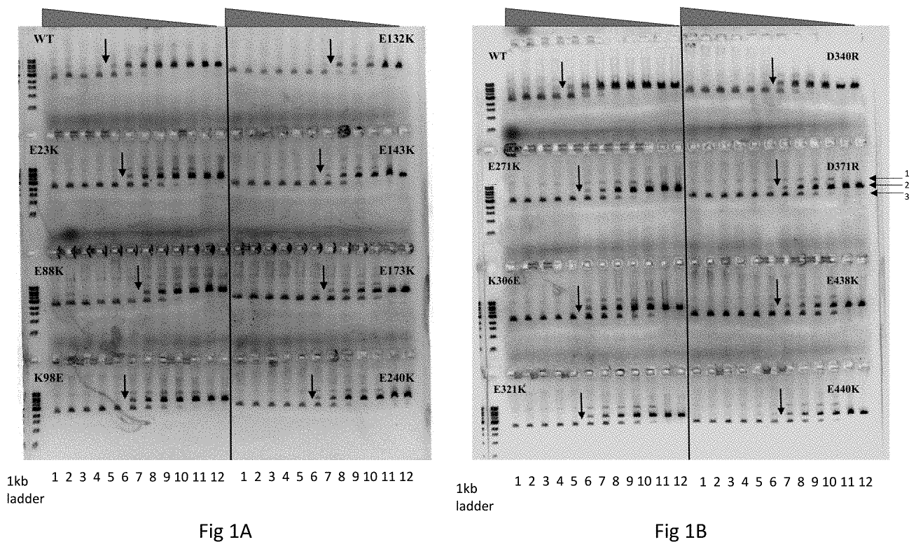

Results as compared with wild type are shown in FIGS. 1A, 1B. The mutants identified that exhibit increased ligation activity are the following: E23K, E88K, K98E, E132K, E143K, E173K, E240K E271K, K306E, E321K, D340R, D371R, E438K, and E440K, as shown in FIGS. 1A, 1B.

Comparative activity results are tabulated in Table 1 below, showing lane differences for each T4 DNA ligase mutant, where each lane difference greater than WT is assigned a 2-fold activity increase. For example, 1 lane activity improvement over WT was given the value 2 (as shown by little or no apparent upper band representing supercoiled plasmid product and little or no apparent middle band representing restriction digested linearized substrate plasmid in a lane), while 2 lanes activity improvement over WT was given the value 4 and so on.

TABLE 1
T4 DNA Ligase variants with greater activity than WT

| | Activity vs. WT |
|---|---|
| Mutants | (Fold) |
| E23K | 2 |
| E88K | 4 |
| K98E | 2 |
| E132K | 8 |
| E143K | 4 |
| E173K | 4 |
| E240K | 2 |
| E271K | 2 |
| K306E | 2 |
| E321K | 2 |
| D340R | 4 |
| D371R | 4 |
| E438K | 4 |
| E440K | 4 |

| T4 DNA Ligase CH Wild Type DNA | | |
|---|---|---|
| SEQ ID NO: 1 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH Wild Type Protein | | |
| SEQ ID NO: 2 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E23K DNA | | |
| SEQ ID NO: 3 | | |
| 1 | ATGATTCTTAAAATTCTGAACGAAATAGCATCTATTGGTTCAACTAAACAGAAGCAAGCA | 60 |
| 61 | ATTCTTAAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E23K Protein | | |
| SEQ ID NO: 4 | | |
| 1 | MILKILNEIASIGSTKQKQAILKKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E88K DNA | | |
| SEQ ID NO: 5 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTAAAGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E88K Protein | | |
| SEQ ID NO: 6 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIKELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH K98E DNA | | |
| SEQ ID NO: 7 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTGAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH K98E Protein | | |
| SEQ ID NO: 8 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGEKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E132K DNA | | |
| SEQ ID NO: 9 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTAAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E132K Protein | | |
| SEQ ID NO: 10 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPKQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E143K DNA | | |
| SEQ ID NO: 11 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATAAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E143K Protein | | |
| SEQ ID NO: 12 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDKKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E173K DNA | | |
| SEQ ID NO: 13 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATAAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E173K Protein | | |
| SEQ ID NO: 14 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDKLDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E240K DNA | | |
| SEQ ID NO: 15 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTAAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E240K Protein | | |
| SEQ ID NO: 16 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPK | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E271K DNA | | |
| SEQ ID NO: 17 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTAAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E271K Protein | | |
| SEQ ID NO: 18 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISKKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH K306E DNA | | |
| SEQ ID NO: 19 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTGAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH K306E Protein | | |
| SEQ ID NO: 20 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSELEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E321K DNA | | |
| SEQ ID NO: 21 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | AAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E321K Protein | | |
| SEQ ID NO: 22 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIKNQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH D340R DNA | | |
| SEQ ID NO: 23 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTCGT | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH D340R Protein | | |
| SEQ ID NO: 24 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIRQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH D371R DNA | | |
| SEQ ID NO: 25 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTCGTGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH D371R Protein | | |
| SEQ ID NO: 26 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIRVDLKIVGIYPHRKDPTKAGGFILESECGKIKVNAGSGLKDKAGVKSHEL | 420 |
| 421 | DRTRIMENQNYYIGKILECECNGWLKSDGRTDYVKLFLPIAIRLREDKTKANTFEDVFGD | 480 |
| 481 | FHEVTGLGSGSSGHHHHHH* | 499 |
| T4 DNA Ligase CH E438K DNA | | |
| SEQ ID NO: 27 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAAAATGCGAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E438K Protein | | |
| SEQ ID NO: 28 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILKCECNGWLKSDGRTDYVKLFLPIAIRLREDKTKANTFEDVFGD | 480 |
| 481 | FHEVTGLGSGSSGHHHHHH* | 499 |
| T4 DNA Ligase CH E440K DNA | | |
| SEQ ID NO: 29 | | |
| 1 | ATGATTCTTAAAATTCTGAACGAAATAGCATCTATTGGTTCAACTAAACAGAAGCAAGCA | 60 |
| 61 | ATTCTTGAAAAGAATAAAGATAATGAATTGCTTAAACGAGTATATCGTCTGACTTATTCT | 120 |
| 121 | CGTGGGTTACAGTATTATATCAAGAAATGGCCTAAACCTGGTATTGCTACCCAGAGTTTT | 180 |
| 181 | GGAATGTTGACTCTTACCGATATGCTTGACTTCATTGAATTCACATTAGCTACTCGGAAA | 240 |
| 241 | TTGACTGGAAATGCAGCAATTGAGGAATTAACTGGATATATCACCGATGGTAAAAAAGAT | 300 |
| 301 | GATGTTGAAGTTTTGCGTCGAGTGATGATGCGAGACCTTGAATGTGGTGCTTCAGTATCT | 360 |
| 361 | ATTGCAAACAAAGTTTGGCCAGGTTTAATTCCTGAACAACCTCAAATGCTCGCAAGTTCT | 420 |
| 421 | TATGATGAAAAAGGCATTAATAAGAATATCAAATTTCCAGCCTTTGCTCAGTTAAAAGCT | 480 |
| 481 | GATGGAGCTCGGTGTTTTGCTGAAGTTAGAGGTGATGAATTAGATGATGTTCGTCTTTTA | 540 |
| 541 | TCACGAGCTGGTAATGAATATCTAGGATTAGATCTTCTTAAGGAAGAGTTAATTAAAATG | 600 |
| 601 | ACCGCTGAAGCCCGCCAGATTCATCCAGAAGGTGTGTTGATTGATGGCGAATTGGTATAC | 660 |
| 661 | CATGAGCAAGTTAAAAAGGAGCCAGAAGGCCTAGATTTTCTTTTTGATGCTTATCCTGAA | 720 |
| 721 | AACAGTAAAGCTAAAGAATTCGCCGAAGTAGCTGAATCACGTACTGCTTCTAATGGAATC | 780 |
| 781 | GCCAATAAATCTTTAAAGGGAACCATTTCTGAAAAAGAAGCACAATGCATGAAGTTTCAG | 840 |
| 841 | GTCTGGGATTATGTCCCGTTGGTAGAAATATACAGTCTTCCTGCATTTCGTTTGAAATAT | 900 |
| 901 | GATGTACGTTTTTCTAAACTAGAACAAATGACATCTGGATATGATAAAGTAATTTTAATT | 960 |
| 961 | GAAAACCAGGTAGTAAATAACCTAGATGAAGCTAAGGTAATTTATAAAAAGTATATTGAC | 1020 |
| 1021 | CAAGGTCTTGAAGGTATTATTCTCAAAAATATCGATGGATTATGGGAAAATGCTCGTTCA | 1080 |
| 1081 | AAAAATCTTTATAAATTTAAAGAAGTAATTGATGTTGATTTAAAAATTGTAGGAATTTAT | 1140 |
| 1141 | CCTCACCGTAAAGACCCTACTAAAGCGGGTGGATTTATTCTTGAGTCAGAGTGTGGAAAA | 1200 |
| 1201 | ATTAAGGTAAATGCTGGTTCAGGCTTAAAAGATAAAGCCGGTGTAAAATCGCATGAACTT | 1260 |
| 1261 | GACCGTACTCGCATTATGGAAAACCAAAATTATTATATTGGAAAAATTCTAGAGTGCAAA | 1320 |
| 1321 | TGCAACGGTTGGTTAAAATCTGATGGCCGCACTGATTACGTTAAATTATTTCTTCCGATT | 1380 |
| 1381 | GCGATTCGTTTACGTGAAGATAAAACTAAAGCTAATACATTCGAAGATGTATTTGGTGAT | 1440 |
| 1441 | TTTCATGAGGTAACTGGTCTAGGTTCTGGCAGTTCAGGTCATCACCACCATCATCACTAA | 1500 |
| T4 DNA Ligase CH E440K Protein | | |
| SEQ ID NO: 30 | | |
| 1 | MILKILNEIASIGSTKQKQAILEKNKDNELLKRVYRLTYSRGLQYYIKKWPKPGIATQSF | 60 |
| 61 | GMLTLTDMLDFIEFTLATRKLTGNAAIEELTGYITDGKKDDVEVLRRVMMRDLECGASVS | 120 |
| 121 | IANKVWPGLIPEQPQMLASSYDEKGINKNIKFPAFAQLKADGARCFAEVRGDELDDVRLL | 180 |
| 181 | SRAGNEYLGLDLLKEELIKMTAEARQIHPEGVLIDGELVYHEQVKKEPEGLDFLFDAYPE | 240 |
| 241 | NSKAKEFAEVAESRTASNGIANKSLKGTISEKEAQCMKFQVWDYVPLVEIYSLPAFRLKY | 300 |
| 301 | DVRFSKLEQMTSGYDKVILIENQVVNNLDEAKVIYKKYIDQGLEGIILKNIDGLWENARS | 360 |
| 361 | KNLYKFKEVIDVDLKIVGIYPHRKDPTKAGGFILESECGKIKVNAGSGLKDKAGVKSHEL | 420 |
| 421 | DRTRIMENQNYYIGKILECKCNGWLKSDGRTDYVKLFLPIAIRLREDKTKANTFEDVFGD | 480 |
| 481 | FHEVTGLGSGSSGHHHHHH* | 499 |

SEQ ID NO: 31 is the recognition sequence GGTCTC for cleavage by the enzyme BsaI-HF® v2

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, including but not limited to Variant Sequences, and that such modifications and variations are considered to be within the scope of this invention as defined by the appended claims.

What is claimed is:

1. A mutant T4 DNA ligase or a biologically active fragment thereof, comprising: one or more of the following the amino acid mutations preceding a comma, wherein the mutation is the substitution at the positions indicated in each amino acid sequence and wherein the entire amino acid sequence is represented in the adjacent even numbered sequence identification number:
(SEQ ID NO: 4)
E23K,
(SEQ ID NO: 6)
E88K,
(SEQ ID NO: 8)
K98E,
(SEQ ID NO: 10)
E132K,
(SEQ ID NO: 12)
E143K,
(SEQ ID NO: 14)
E173K,
(SEQ ID NO: 16)
E240K,
(SEQ ID NO: 18)
E271K,
(SEQ ID NO: 20)
K306E,
(SEQ ID NO: 22)
E321K,
(SEQ ID NO: 24)
D340R,
(SEQ ID NO: 26)
D371R,
(SEQ ID NO: 28)
E438K
and
(SEQ ID NO: 30)
E440K.

2. A mutant T4 DNA ligase or a biologically active fragment thereof of claim 1 but wherein each said amino acid sequence of the corresponding mutant T4 DNA ligase does not include the 6-membered histidine tag at its C-terminus and the eight immediately preceding Glycine, Leucine and Serine amino acid residues in each said even numbered sequence identification number.

3. A polynucleotide encoding an amino acid sequence of any one of the mutant T4 DNA ligases of claim 1.

4. A polynucleotide encoding an amino acid sequence of any one of the mutant T4 DNA ligases of claim 2.

5. A mutant T4 DNA ligase or a biologically active fragment thereof, comprising: one or more of the following the amino acid mutations preceding a comma, wherein the mutation is the substitution at the positions indicated in each amino acid sequence and wherein the entire amino acid sequence is represented in the adjacent even numbered sequence identification number:
(SEQ ID NO: 4)
E23K,
(SEQ ID NO: 6)
E88K,
(SEQ ID NO: 8)
K98E,
(SEQ ID NO: 10)
E132K,
(SEQ ID NO: 12)
E143K,
(SEQ ID NO: 14)
E173K,
(SEQ ID NO: 16)
E240K,
(SEQ ID NO: 18)
E271K,
(SEQ ID NO: 20)
K306E,
(SEQ ID NO: 22)
E321K,
(SEQ ID NO: 24)
D340R,
(SEQ ID NO: 26)
D371R,
(SEQ ID NO: 28)
E438K
and
(SEQ ID NO: 30)
E440K;
and
wherein each said amino acid sequence has conservative substitutions for certain of its amino acids but only to the extent that there remains at least 70% sequence identity to the sequence represented in the adjacent even numbered sequence identification number.

6. A mutant T4 DNA ligase or a biologically active fragment of claim 5, but wherein each said amino acid sequence of the corresponding mutant T4 DNA ligase does not include the 6-membered histidine tag at its C-terminus and the eight immediately preceding Glycine, Leucine and Serine amino acid residues in each said even numbered sequence identification number, and wherein there remains at least 70% sequence identity to the sequence represented in the adjacent even numbered sequence identification number without the 6-membered histidine tag at its C-terminus and the eight immediately preceding Glycine, Leucine and Serine amino acid residues in each said even numbered sequence identification number.

7. A mutant T4 DNA ligase of claim 6 wherein each said amino acid sequence has conservative substitutions for certain of its amino acids but only to the extent that there remains at least 80%, 90%, 95%, 96%, 97%, 98% or 99% sequence identity to the sequence represented in the adjacent even numbered sequence identification number without the 6-membered histidine tag at its C-terminus and the eight immediately preceding Glycine, Leucine and Serine amino acid residues.

8. A polynucleotide encoding an amino acid sequence of one of the mutant T4 DNA ligases of claim 5.

9. A polynucleotide encoding an amino acid sequence of one of the mutant T4 DNA ligases of claim 6.

10. A polynucleotide encoding an amino acid sequence of one of the mutant T4 DNA ligases of claim 7.

11. A polynucleotide of claim 1 having one of the following DNA sequences:
(SEQ ID NO: 3)
E23K,
(SEQ ID NO: 5)
E88K,
(SEQ ID NO: 7)
K98E,
(SEQ ID NO: 9)
E132K,
(SEQ ID NO: 11)
E143K,
(SEQ ID NO: 13)
E173K,
(SEQ ID NO: 15)
E240K,
(SEQ ID NO: 17)
E271K,
(SEQ ID NO: 19)
K306E,
(SEQ ID NO: 21)
E321K,
(SEQ ID NO: 23)
D340R,
(SEQ ID NO: 25)
D371R,
(SEQ ID NO: 27)
E438K
and
(SEQ ID NO: 29)
E440K.

12. A polynucleotide of claim 11 wherein said polynucleotide does not include the 144 C-terminal nucleotides of each of said following DNA sequences.

13. A vector incorporating a polynucleotide of claim 12.

14. A vector incorporating a polynucleotide of claim 9.

15. A cell transformed with and expressing a polynucleotide of claim 13.

16. A cell transformed with and expressing a polynucleotide of claim 9.

17. A process of conducting polynucleotide ligation between different polynucleotides or by ligating the 5′ and 3′ ends of polynucleotides to generate circular polynucleotide, wherein the polynucleotides have blunt ends or cohesive ends, comprising:
providing a ligation mixture including the polynucleotides to be ligated and a mutant T4 DNA ligase or a biologically active fragment of claim 2; and
placing said ligation mixture at temperatures wherein ligation takes place.

18. A process of conducting polynucleotide ligation between different polynucleotides or by ligating the 5′ and 3′ ends of polynucleotides to generate circular polynucleotide, wherein the polynucleotides have blunt ends or cohesive ends, comprising:
providing a ligation mixture including the polynucleotides to be ligated and a mutant T4 DNA ligase or a biologically active fragment of claim 1; and
placing said ligation mixture at temperatures wherein ligation takes place.

19. A process of conducting polynucleotide ligation between different polynucleotides or by ligating the 5′ and 3′ ends of polynucleotides to generate circular polynucleotide, wherein the polynucleotides have blunt ends or cohesive ends, comprising:
providing a ligation reaction mixture including a buffer, the polynucleotides to be ligated and a mutant T4 DNA ligase or a biologically active fragment of claim 6; and
placing said ligation reaction mixture under temperature conditions suitable for ligation.

20. The process of claim 18 wherein the ligation reaction mixture includes Tris-HCl, MgCl₂, ATP, dithiothreitol and water.
